(19) United States
(12) Patent Application Publication
(10) Pub. No.: US 2012/0164712 A1
(43) Pub. Date: Jun. 28, 2012

(54) PRODUCTION OF ALGAE

(76) Inventors: Gary Kenneth Ellem, Newcastle (AU); Annelie Karin Moberg, The Hill (AU); Ross Anthony McGregor, Mayfield (AU); Joseph George Herbertson, Toronto (AU)

(21) Appl. No.: 13/377,478

(22) PCT Filed: Jun. 10, 2010
(86) PCT No.: PCT/AU2010/000718

§ 371 Date: Mar. 20, 2012

(30) Foreign Application Priority Data
Jun. 10, 2009 (AU) 2009902648

(51) Int. Cl. C12N 1/12; C12M 1/24

(52) U.S. Cl. 435/257.1; 435/292.1

(57) ABSTRACT

A closed system for producing algae moves water that contains algae through an endless reactor tube that defines a continuous pathway. At least part of the tube floats on a volume of water. The tube is transparent to light. Water partially fills the tube and there is a gas space above the water in the tube, with two phase stratified flow of water and gas in the tube, gas transfer between the gas space and the water, and algae biomass suspended in the water. Nutrients and a gas and water are supplied to the tube. Algae are harvested from the tube.

The present invention relates to the production of algae.

Algae are a valuable source of biomass, and derived products such as oils, protein and biomolecules. However, there are significant issues involved in growing algae in sufficient amounts in a cost effective manner at an industrial scale.

According to the present invention there is provided a method of producing algae in a closed system that comprises:
(a) moving water that contains algae along a continuous pathway in a closed system, with at least part of the pathway being in a reactor tube floating on a volume of water, with the reactor tube being transparent to light, and with water partially filling the tube, and with a gas space above the water in the tube, whereby there is two phase stratified flow of water and gas in the reactor tube, whereby there is gas transfer between the gas space and the water, and whereby there is algae biomass suspended in the water;
(b) circulating the algae suspension in the tube during periods of sunlight to expose algae in the tube to sunlight to facilitate growth of the algae in the tube, and
(c) harvesting algae from the tube.

The term “closed system” is understood herein to mean that the system is isolated from the atmosphere and there is controlled supply of fluids into and discharge of fluids from the system.

The use of the closed system as opposed to an open system has a number of benefits. Productivity in a closed system can be far higher than open systems as a number of variables can be controlled, foremost fluid dynamics, light exposure, nutrient concentration and regulation of temperature, salinity and pH of the culture media. By controlling the fluid dynamics in the system, available light may be fully utilised, thereby optimising production. Moreover, isolating the culture in the closed system carries with it a lower risk of contamination or competition from bacterial, fungal of predatory organisms than is the case with open systems. In addition, the closed system effectively provides a barrier and therefore enables there to be selectivity over a chosen species or group of species to be cultivated.

The method may comprise moving the water that contains algae along an endless pathway and supplying water or nutrient media to and discharging water/algae suspension from the endless pathway at selected locations along the pathway.

The method may comprise floating the partially water filled reactor tube on the volume of water such that the water level in the reactor is approximately level with the surface of the volume of water. In this configuration the algae culture in the reactor tube are always surrounded by a far greater volume of water outside of the tube. This arrangement takes advantage of the thermal mass in the surrounding water to buffer diurnal temperature variation and maintain the temperature in the culture at a level where optimum growth is attained. The flotation of the tube in the volume of water may be mainly achieved through the inherent buoyancy of the system through the gas space along the length of the reactor tube, particularly when the air space is under positive pressure. It may also be achieved by any one or more of materials selection to control the weight of the system, controlling the pressure in the air space, and the use of buoyancy to support the system.

The floating tube may comprise at least 70%, typically at least 80%, of the length of the pathway.

The method may comprise maintaining the water level in the tube at 40-60%, typically 45-55%, of the volume of the tube. Maintaining the water level within this range ensures that there is a large surface area at the interface between the water and the gas space. This allows release of O₂ from the water and replenishment of CO₂ in the water during photosynthesis in periods of sunlight, and the replenishment of O₂ during periods of darkness where O₂ concentrations may fall.

The method may comprise maintaining an over-pressure in the gas space. Maintaining the over-pressure provides a level of rigidity of the tubes—this is important when the tubes are made from flexible material that has no inherent structural rigidity. The over-pressure also contributes to buoyancy when the tube is located in a volume of water.

The method may comprise moving gas in the gas space in the tube.

The method may comprise supplying a gas into the gas space and discharging gas from the gas space.

The gas may contain CO₂ and O₂. The method may comprise controlling the concentrations of CO₂ and O₂ in the gas to regulate photosynthesis and respiration during daytime and nighttime periods, respectively.

The gas supplied to the gas space may be selected to promote photosynthesis in the algae during periods of sunlight. The gas may also be selected to support respiration in algae during periods of nighttime. Typically, the gas has higher concentrations of CO₂ and lower concentrations of O₂ during periods of sunlight than during periods of nighttime.

The gas supplied to the gas space may be supplied co-current or counter-current to the flow of water and algae in the tube.

The method may comprise controlling the fluid dynamics of the culture in the system to utilize the self-shading effect of a dense culture so that algae cells are continuously moving between darker and lighter zones of the culture (under the influence of the preferably turbulent flow regime inside the reactor tube), creating a light-dark cycle pattern that may promote algae growth. The ratio of the light zone to the dark zone may be regulated in relation to the incident light intensity by the concentration of the algae in the culture. Partially shielding algae cells from intense light in this manner makes it possible to avoid overexposure of algae to sunlight.

The control of light exposure, nutrient availability and mass transfer of gasses may be achieved by the fluid dynamics of circulating the algae culture in the tube.

The pathway may comprise a vertical section, and the method may comprise transporting water through the pathway using a gas uplift pump that injects a gas into the vertical section to create an uplift effect that causes water in the vertical section to flow in the direction of the bubbles.

One advantage of the gas uplift pump is that circulation of algae and the required supply of CO₂ and O₂ to the water may be achieved by a single input. For example, the gas may be selected to promote photosynthesis in the algae during periods of sunlight. The gas may also be selected to promote respiration in algae during periods of nighttime. Typically, the gas has higher concentrations of CO₂ and lower concentrations of O₂ during periods of sunlight than during periods of nighttime.

The method may comprise controlling the concentrations of CO₂ and O₂ in the gas injected into the water via the gas uplift pump to promote photosynthesis and respiration during daytime and nighttime periods, respectively.

Another advantage of this gas uplift pump is that the force generated by the gas injection is inherently low shear. Low shear is preferable to high shear in most algal applications as many algae species are sensitive to shear, performing sub-optimally under such conditions.

The method may comprise circulating algae in the tube by pump options other than the above-described gas uplift pump. Other options include, by way of example, screw, peristaltic, centrifugal, or impeller design pumps. These other options may not require providing the pathway with a vertical section. Hence, in its most simplest form the tube may be a horizontally-disposed loop that defines the endless pathway and is partially filled with algae culture and includes water or nutrient media inlets and water/algae suspension outlets from the endless pathway at selected locations along the pathway.

The method may support high density growth and may comprise maintaining a concentration of algae in the tube greater than 1 gram per litre or greater than 10 grams per litre.

A degree of control over flow rates may be achieved by manipulating the amount of gas supplied to the system. An increase in gas flow into the system corresponds with an increase in fluid flow around the tube, an increase in gas and temperature exchange between the closed system and the atmosphere outside the tube and an increase in turbulence within the algae in the tube.

The method may be carried out on a batch basis or on a continuous basis.

In a batch basis operation, all of the algae is harvested after a period of time.

In a continuous basis operation, the algae may be harvested periodically or continuously.

The method may recycle the water media back into the reactor or discharge it as required.

According to the present invention there is provided an apparatus for producing algae in a closed system that comprises:
(a) a photobioreactor that defines a continuous pathway in the closed system, the photobioreactor comprising a reactor tube that has a section that contains algae culture and gas in a gas space above the algae culture and can float on a volume of water, the section of the tube defining a photoactive part of the pathway,
(b) a means for moving the water in the tube along the continuous pathway;
(c) a means for controlling the flow conditions in the tube to induce flow of the algae culture in the tube as required to facilitate growth of the algae;
(d) an inlet for supplying a gas to the gas space,
(e) an outlet for discharging gas from the gas space;
(f) an inlet for water or nutrient media, and
(g) an outlet for water/algae suspension.

The tube of the photobioreactor may define an endless loop that forms the continuous pathway and include the gas inlet, the gas outlet, the water or nutrient media inlet, and the water/algae suspension outlet at selected locations along the tube.

The tube of the photobioreactor may be a continuous vertically-disposed loop that can float on the volume of water, with the gas inlet, the gas outlet, the water or nutrient media inlet, and the water/algae suspension outlet at selected locations along the tube.

The tube of the photobioreactor may be a continuous horizontally-disposed loop that can float on the volume of water, with the gas inlet, the gas outlet, the water or nutrient media inlet, and the water/algae suspension outlet at selected locations along the tube.

The apparatus may comprise a plurality of the above-described photobioreactors, a framework for physically connecting the photobioreactors together, and a network of plumbing for supplying the gas and the water or nutrient media inlet to each bioreactor and for removing the gas and the water/algae suspension from each bioreactor.

The buoyancy of the partially filled section of tube means that the tube can float with the water surface in the tube being approximately equal to the water level of the supporting water volume. As is described above, the buoyancy may be mainly due to the gas space in the partially filled tube, particularly when the gas space is pressurized. In addition, as is described above, there may be other factors that contribute to different extents to the buoyancy of the tube.

The floating tube of the bioreactor may comprise at least 70%, typically at least 80%, of the length of the pathway.

The water level in the tube may be 40-60%, typically 45-55%, of the volume of the tube.

The fact that the pathway comprises a floating tube section is particularly important when the tube is formed from a flexible material, such as a polymeric material, that has little physical rigidity of itself so that the water allows the otherwise flexible tube to form a long tubular reactor space without any requirement for a rigid structure. While there is no requirement for use of rigid materials in construction of the reactor space, rigid materials may be used if desired.

Typically, the tube is formed from a transparent material.

The tube may be made from a flexible material, such as a polymeric material, that is transparent to visible and infra red light, typically with a thickness greater than 50 micrometres and less than 1,000 micrometres, more typically between 250 micrometres and 750 micrometres.

The tube may be made from polyethylenes. Polyethylenes are advantageous materials because of availability in large quantities.

The tube may be any suitable cross-section.

The tube may have a circular cross-section.

The tube may be at least 10 m long.

The tube may also be at least 50 m long.

The apparatus may comprise a pump, such as a gas uplift pump, typically an air lift pump, to move water and algae through the tube.

The pathway may comprise a vertical section.

The gas uplift pump may be positioned to inject a gas into the vertical section to create an uplift effect that causes water in the vertical section to flow in the direction of the bubbles.

The photobioreactor may comprise any suitable arrangement of tubes that define one or more than one continuous or discontinuous pathway.

The photobioreactor may comprise any suitable arrangement of tubes that define a flow through pathway.

According to the present invention there is provided a bioreactor system that comprises the above-described apparatus positioned in a volume of water.

The volume of supporting water may be selected to be at least sufficient to act as a thermal mass to facilitate temperature control of the bioreactor.

The present invention is independent of algae type in the sense that the method and the apparatus may be adapted to operate with many algae species, typically planktonic microalgae.

The present invention is described further by way of example with reference to the accompanying Figures, of which:

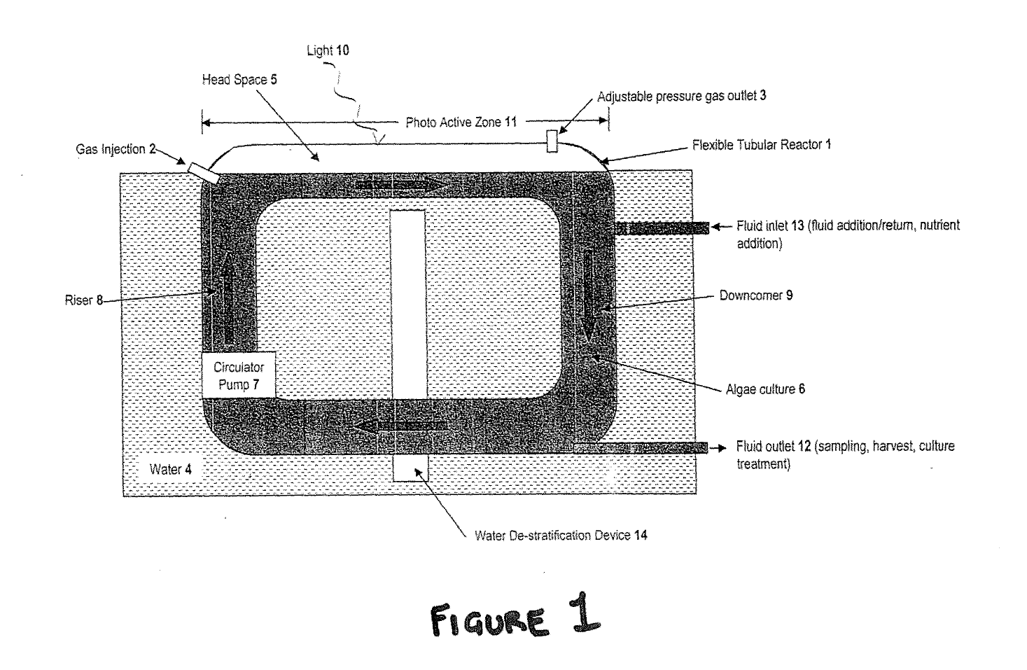

FIG. 1 is a diagrammatic side elevation of one embodiment of an apparatus in accordance with the present invention, in an operational state floating in a volume of water; and

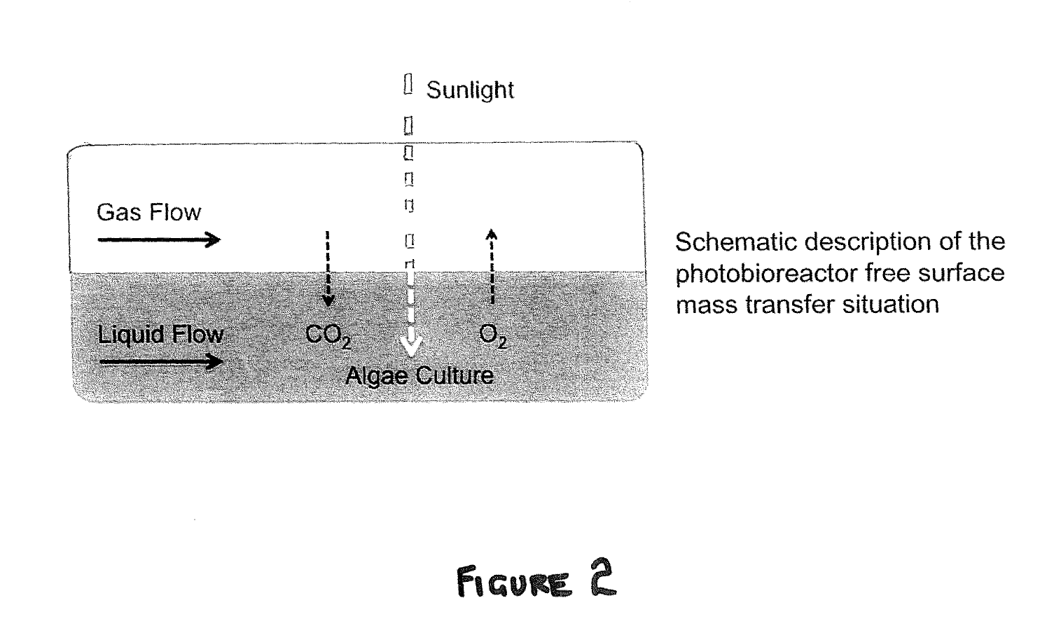

FIG. 2 is a schematic description of the free surface gas transfer in the apparatus shown in FIG. 1.

The Figures show a closed system for producing algae.

In general terms, the system shown in the Figures moves water, with algae suspended in the water, through an endless reactor tube that defines a continuous pathway. At least part of the tube floats on a volume of water. The tube is transparent to light. Water partially fills the tube and there is a gas space above the water in the tube, with two phase stratified flow of water and gas in the tube, gas transfer between the gas space and the water across the free surface between these phases, and algae biomass suspended in the water. Nutrients and a suitable gas and water are supplied to the tube to promote photosynthesis of algae. Algae are harvested from the tube. The stratified flow and the gas transfer at the free surface between the gas space and the water are shown in FIG. 2.

In more specific terms, the apparatus shown in FIG. 2 comprises a flexible tubular bioreactor (1) that comprises an endless, vertically-disposed tube that is partially filled with algae culture and has a gas space (5) above the level of the water in a horizontal upper section of the tube. The tube is constructed of thin transparent material with no inherent structural rigidity such as polyethylene or PVC and without the requirement for external rigid structures. The tube defines a continuous pathway for two phase stratified flow of algae culture and gas within the tube. Tube sections are formed either through blow forming of plastic sleeve or by strip welding two layers of sheet material together. The diameter of the tube sections may be any suitable diameter. Critically shaped regions such as corners or connections are welded into the same plastic or formed from blow or injection moulded material that are attached to the tubular regions.

FIG. 2 shows the apparatus very diagrammatically and, by way of example, practical embodiments of the apparatus may have partially filled floating tube sections that are at least 10 m long and vertical tube sections that are typically more than 5% of the horizontal length. The horizontal underwater water filled tube section typically has a length as close as practicable to zero.

Shape rigidity of the reactor is developed by providing pressure inside the reactor by pressurised gas injection (2) and back pressure developed by a gas outlet (3). Examples of devices for back pressure development are flow restrictors, tensioned valves and fluid columns.

The reactor shape is achieved at minimal pressure by floating the reactor in a volume of water (4). Buoyancy is achieved via a predetermined gas volume in the gas space (5) and stability may be enhanced by flotation devices attached to the reactor. In this region the gas and fluid volumes are ideally equal such that the tube is half full of fluid medium. Smaller tube diameters are able to withstand higher reactor pressures for the same material use and have a higher light exposure per unit of reactor volume. Tube diameter influences the fluid flow regime within the reactor.

The algae culture (6) is circulated around the tubular loop by a circulator pump (7). Examples of the pump are gas uplift, screw, peristaltic, centrifugal, or impeller design. If configured as a gas uplift pump with gas injection in a vertical section of the tube, the gas injection (2) may be used as the gas source to promote photosynthesis of algae during periods of sunlight and respiration of algae during nighttime periods. Fluid velocity within the reactor can be altered by the pumping rate.

Gas flow rate in the gas space (5) is a function of the gas injection rate. The direction of gas flow in the head space may be con-current or counter-current to the direction of water and algae flow depending on the placement of gas injection and outlet ports. Multiple injection and outlet ports may be configured if desired. Outlet gases may be recycled through a compressor to the input if required.

Gas exchange between the gas space (5) and culture medium is controlled via the injection gas composition and the velocities of the fluid and gas phases.

Light exposure of algae cells is a function of the intensity of incident light (10), absorbance of the algae culture, length of the light path resulting from the tube diameter, culture depth and cell density, and turbulent mixing as a result of the fluid velocity in the photoactive zone (11). The efficiency and rate of reactor productivity for a given tube diameter in relation to the available incident light can therefore be optimised by adjustment of the injected gas composition and rate as well as the circulating fluid velocity and culture density. The adjustment variables may be set in a single position for static artificial lighting conditions, or controlled in real time in response to variation in the incidence of natural light.

Water/culture removal for harvest, sampling, treatment or system maintenance is via the fluid outlet (12). Fluid exits under the positive pressure of the reactor, but may be assisted by a fluid pump if required. Fluid return and addition of water, nutrient or treatment chemicals occurs via the fluid inlet (13). Inputs via the fluid inlet require an input pump if the pressure is below that of the reactor.

Temperature fluctuations within the reactor are minimised via the thermal mass of the supporting water body. Heat stratification of the water body is likely in cases where there is little mixing of the supporting water body. This may be used to raise the reactor temperature in relation to the supporting water column if required, or a de-stratification device (14) may be used to reduce the reactor temperature to the vicinity of the bulk water temperature. Examples of de-stratification devices are fluid mixing devices such as impellers, bubblers and air lifters, or heat transfer devices such as conductors, heat pumps or heat transfer engines.

The buoyancy of the half full photo active zone means that the upper surface of the tube is not continuously contacted by the culture media or external water body. This limits bio-fouling and permits extended operation without the requirement for internal or external tube cleaning.

In terms of scale-up, the apparatus may comprise a plurality of the bioreactors (1) connected together by a suitable framework, and plumbing to supply gas, water and nutrients to each bioreactor and to remove water/algae suspension from each bioreactor.

### EXAMPLES

The applicant has carried out a series of experiments to evaluate the method and the apparatus of the present invention.

The experiments were carried out on prototypes having the basic features of the apparatus shown in FIG. 2.

The experiments were carried out using a single culture of Tetraselmis Chuii and a mixed culture of Tetraselmis Chuii, Isochrysis galbana and Chaetoceros muelleri.

### Prototype 1

A very basic form of the apparatus shown in FIG. 2 was made from readily available plumbing fittings and flexible tubes.

### Conclusions in Relation to Prototype 1

Prototype 1 was an achievement of a stable robust system that supported a live algae culture. The action of the air lift pumps was confirmed. Alignment of the airlift pump at the water line was stable with or without air flow. A tube size of 110 mm diameter was used in this case. Half filled tubes sat at the surface of the sea water pond given the correct volume of water in the reactor.

The system was functional at a wide range of internal water salinities from fresh to that more saline than the supporting pond. A reasonable level of flow and turbulence was observed and a reasonable level of isolation of the culture was possible, enough to begin trials of specific live cultures.

### Prototype 2

For prototype 2, both major and minor revisions of basic design principles were considered. Six identical systems were built to start some replicated trials with live cultures testing variables like nutrient status, density optimums and the effect of varied salinities on living cultures.

### Conclusions for Prototype 2

A significant amount of the initial design elements from prototype 1 were carried across to prototype 2 and the modifications made to Prototype 1 were successful and further confirmed the potential of the invention.

### Prototype 3

Prototype 3 was a modular unit where the photoactive zone was of 25 m² horizontal light exposed area. The unit was built in a way that could be scaled in either direction. The modular unit was built as a direct extrapolation of the prototype 1 and 2 designs. Slightly different plumbing was employed in some respects although the plumbing closely followed the operation of the prototype 1 design. The unit was designed to be able to transfer part of the algae culture to shore, in a parallel loop, continuously or discontinuously during the normal function of the system. This made it possible to sample, harvest and return water to the system without reversing any of the plumbing flows. Behaviours of the system that differed from previous designs were observed to confirm that all the past work on the smaller prototypes was relevant to the new system. Differences in operating procedures or culture performance were investigated to see if they could be justified in terms of design.

### Conclusions in Relation to Prototype 3

With prototype 3 it was clear that the basis design was stable and reliable, capable of supporting a live algae culture for extended periods (approximately 3 months in this trial). The basic functions of the system were found to be secure; the system could reliably circulate culture at a variable rate around the photoactive parts of the system. The plumbing to and from the system allowed movement of culture around the system and harvest loops with isolation from the external environment. The system could be drawn from or added to without reversing any of the running state flows.

Many modifications may be made to the embodiment of the method and apparatus of the present invention described above without departing from the spirit and scope of the invention.

1. A method of producing algae in a closed system that comprises:
(a) moving water that contains algae along a continuous pathway in a closed system, with at least part of the pathway being in a reactor tube floating on a volume of water, with the reactor tube being transparent to light, and with water partially filling the tube, and with a gas space above the water in the tube, whereby there is two phase stratified flow of water and gas in the reactor tube, whereby there is gas transfer between the gas space and the water, and whereby there is algae biomass suspended in the water;
(b) circulating the algae suspension in the tube during periods of sunlight to expose algae in the tube to sunlight to facilitate growth of the algae in the tube, and
(c) harvesting algae from the tube.

2. The method defined in claim 1 comprises moving the water that contains algae along an endless pathway and supplying water or nutrient media to and discharging water/algae suspension from the endless pathway at selected locations along the pathway.

3. The method defined in claim 1 comprises floating the partially water filled reactor tube on the volume of water such that the water level in the reactor is approximately level with the surface of the volume of water.

4. The method defined in claim 1 comprises maintaining the water level in the tube at 40-60% of the volume of the tube.

5. The method defined in claim 1 comprises maintaining an over-pressure in the gas space

6. The method defined in claim 1 comprises moving gas in the gas space in the tube.

7. The method defined in claim 1 comprises supplying a gas into the gas space and discharging gas from the gas space.

8. The method defined in claim 7 wherein the gas contains CO₂ and O₂ and the method comprises controlling the concentrations of CO₂ and O₂ in the gas to regulate photosynthesis and respiration during daytime and nighttime periods, respectively.

9. The method defined in claim 8 comprises selecting higher concentrations of CO₂ and lower concentrations of O₂ during periods of sunlight than during periods of nighttime.

10. The method defined in claim 1 comprises controlling the fluid dynamics of the culture in the system to utilize the self-shading effect of a dense culture so that algae cells are continuously moving between darker and lighter zones of the culture, creating a light-dark cycle pattern that may promote algae growth.

11. The method defined in claim 1 wherein the pathway comprises a vertical section, and the method comprises transporting water through the pathway using a gas uplift pump that injects a gas into the vertical section to create an uplift effect that causes water in the vertical section to flow in the direction of the bubbles.

12. The method defined in claim 11 comprises selecting the gas to promote photosynthesis in the algae during periods of sunlight.

13. The method defined in claim 11 comprises selecting the gas to promote respiration in algae during periods of nighttime.

14. The method defined in claim 11 comprises controlling the concentrations of CO₂ and O₂ in the gas injected into the water via the gas uplift pump to promote photosynthesis and respiration during daytime and nighttime periods, respectively.

15. An apparatus for producing algae in a closed system that comprises
(a) a photobioreactor that defines a continuous pathway in the closed system, the photobioreactor comprising a reactor tube that has a section that contains algae culture in water and gas in a gas space above the algae culture and and can float on a volume of water, the section of the tube defining a photoactive part of the pathway,
(b) a means for moving the water in the tube along the continuous pathway;
(c) a means for controlling the flow conditions in the tube to induce flow of the algae culture in the tube as required to facilitate growth of the algae;
(d) an inlet for supplying a gas to the gas space,
(e) an outlet for discharging gas from the gas space;
(f) an inlet for water or nutrient media, and
(g) an outlet for water/algae suspension.

16. The apparatus defined in claim 15 wherein the tube of the photobioreactor defines an endless loop that forms the continuous pathway and includes the gas inlet, the gas outlet, the water or nutrient media inlet, and the water/algae suspension outlet at selected locations along the tube.

17. The apparatus defined in claim 15 wherein the tube of the photobioreactor is a continuous vertically-disposed loop that can float on the volume of water, with the gas inlet, the gas outlet, the water or nutrient media inlet, and the water/algae suspension outlet being at selected locations along the tube.

18. The apparatus defined in claim 15 wherein the tube of the photobioreactor is a continuous horizontally-disposed loop that can float on the volume of water, with the gas inlet, the gas outlet, the water or nutrient media inlet, and the water/algae suspension outlet being at selected locations along the tube.

19. The apparatus defined in claim 15 comprises a plurality of the photobioreactors, a framework for physically connecting the photobioreactors together, and a network of plumbing for supplying the gas and the water or nutrient media inlet to each bioreactor and for removing the gas and the water/algae suspension from each bioreactor.

20. The apparatus defined in claim 15 wherein the floating tube of the bioreactor comprises at least 70% of the length of the pathway.

21. The apparatus defined in claim 15 wherein the water level in the tube is 40-60% of the volume of the tube.

22. The apparatus defined in claim 15 comprises a pump to move water and algae through the tube.

23. The apparatus defined in claim 15 wherein the pathway comprises a vertical section and the apparatus comprises a gas uplift pump positioned to inject a gas into the vertical section to create an uplift effect that causes water in the vertical section to flow in the direction of the bubbles.

24. A bioreactor system that comprises the apparatus defined in claim 15 positioned in a volume of water.
